(19) United States
(12) Patent Application Publication
(10) Pub. No.: US 2011/0311557 A1
(43) Pub. Date: Dec. 22, 2011

(54) RECIPROCAL SERUM/PLASMA EXCHANGE FOR THE TREATMENT OF CANCER

(76) Inventor: Martin Heath Bluth, Southfield, MI (US)

(21) Appl. No.: 13/148,575

(22) PCT Filed: Feb. 9, 2010
(86) PCT No.: PCT/US2010/023584

§ 371 Date: Aug. 9, 2011

Related U.S. Application Data
(60) Provisional application No. 61/151,693, filed on Feb. 11, 2009.

(51) Int. Cl. A61K 39/395; A61P 35/00; A61P 37/04

(52) U.S. Cl. 424/174.1; 424/130.1

(57) ABSTRACT

Techniques for treating cancer are provided. The techniques include obtaining an amount of serum and/or plasma from at least a first patient with a given type of cancer and administering at least a portion of the serum and/or plasma obtained from the first patient to at least a second patient with the same or different type of cancer. Administering the serum and/or plasma obtained from the first patient to the second patient includes administering a therapeutically effective amount of one or more immunoglobulins and one or more soluble receptors corresponding to each immunoglobulin so as to enable reconstitution of a humoral immune system of the second patient to thereby treat the cancer in the second patient.

### CROSS-REFERENCE TO RELATED APPLICATION(S)

This application claims the benefit of U.S. provisional application Ser. No. 61/151,693, filed on Feb. 11, 2009, the complete disclosure of which is expressly incorporated herein by reference in its entirety for all purposes. This application is related to pending PCT Application No. PCT/US08/063,895, filed on May 16, 2008, which claims priority to U.S. Provisional Application Ser. No. 60/938,753, filed on May 18, 2007, the disclosures of which are expressly incorporated by reference herein in their entireties for all purposes.

### FIELD OF THE INVENTION

The present invention relates generally to immunology and, more particularly relates to diagnosis and treatment of cancer.

### BACKGROUND OF THE INVENTION

Cancer, otherwise known as malignancy, is a devastating disease that is challenging to treat and, in certain cases, offers little chance of survival. By way of example, pancreatic cancer, as a representation of cancer, carries a dismal prognosis and high mortality rate. Despite existing therapeutic approaches such as, for example, gemcitabine (e.g., Gemzar®, a registered trademark of Eli Lilly and Company) and anti-topoisomerases, pancreatic cancer remains among the ten leading causes of death due to cancer in the United States.

Early diagnosis of certain cancers (for example, pancreatic cancer) can be extremely difficult, as existing approaches include imaging techniques with subsequent histopathological confirmation from biopsy specimens. In many cases, patients present in late stages of the disease and are no longer amenable to surgery or treatment.

Existing diagnostic approaches demonstrate that immunoglobulin levels can be correlated with disease severity. By way of example, in pancreatic cancer, pancreatic fluid obtained from patients with pancreatic cancer have been assayed and shown to have increased levels of immunoglobulin G (IgG) and immunoglobulin A (IgA), with borderline increases in immunoglobulin M (IgM) when compared with healthy controls.

Additionally, existing diagnostic approaches include postmortem examination of the pancreas and other organs from patients with adenocarcinoma, which demonstrated IgA and some IgM deposits, as did pancreato-duodenal fluid obtained from living patients.

Other existing approaches for immunological strategies have been pursued. For example, stimulation of the patient's own immune system to fight their malignancy has been attempted with cytokines such as, for example, GM-CSF, IL-6, and immune response modifiers such as, for example, Virulizin® (a registered trademark of Lorus Therapeutics, Inc.). However, conventional approaches to diagnosing and treating many malignancies such as pancreatic cancer have, thus far, been largely unsuccessful.

### SUMMARY OF THE INVENTION

The present invention, in illustrative embodiments thereof, provides techniques for the treatment of cancer (e.g., pancreatic cancer).

In accordance with one aspect of the invention, an exemplary method for treating cancer includes the steps of: obtaining an amount of serum and/or plasma from at least a first patient with a given type of cancer; and administering at least a portion of the serum and/or plasma obtained from the first patient to at least a second patient with the same or a different type of cancer. Administering the serum and/or plasma obtained from the first patient to the second patient includes administering a therapeutically effective amount of one or more immunoglobulins and one or more soluble receptors corresponding to each immunoglobulin so as to enable reconstitution of a humoral immune system of the second patient to thereby treat the cancer in the second patient.

Preferably, a corresponding amount of serum and/or plasma is removed from the second patient, either prior to or substantially simultaneous with administering the serum and/or plasma from the first patient to the second patient.

This approach can be repeated and administered in tandem to other patients with the same or different types of cancer. For example, serum and/or plasma can be obtained from a third or more patient(s) and administered to the first patient in no particular set sequence. Such administration can be administered as a single dose or as repeated doses. In accordance with aspects of the invention, serum and plasma may be considered equivalent to one another, at least with respect to therapy.

These and other features, objects and advantages of the present invention will become apparent from the following detailed description of illustrative embodiments thereof, which is to be read in connection with the accompanying drawings.

### BRIEF DESCRIPTION OF THE DRAWINGS

FIG. 1 is a diagram illustrating serum levels of IgG, IgM and IgA in samples with pancreatic cancer versus healthy control samples without pancreatic cancer, according to an embodiment of the present invention;

FIG. 2 is a diagram illustrating serum levels of IgE and soluble CD23 (sCD23) in samples with pancreatic cancer versus healthy control samples without pancreatic cancer, according to an embodiment of the present invention;

FIG. 3 is a diagram illustrating presence of pancreatic cancer-specific immunoglobulins by immunofluorescence, according to an embodiment of the present invention;

FIG. 4 is a diagram illustrating antigen-specific antibodies, according to an embodiment of the present invention;

FIG. 5 is a diagram illustrating IgE mediated ADCC and effect of neutralization, according to an embodiment of the present invention;

FIG. 6 is a diagram illustrating IgE mediated ADCC and effect of IgE depletion and purified IgE; and

FIG. 7 is a flow diagram illustrating techniques for treating cancer in a patient, according to an embodiment of the present invention.

### DETAILED DESCRIPTION OF PREFERRED EMBODIMENTS

The present invention, in illustrative embodiments thereof, provides techniques for the treatment of cancer in a patient. The term “patient” as used herein is intended to refer broadly to mammalian subjects, preferably humans, receiving medical attention (e.g., diagnosis, monitoring, etc.), care or treatment. Also, a “therapeutically effective amount” of a given compound in a treatment methodology may be defined herein as an amount sufficient to produce a measurable attenuation and/or a measurable diagnostic effect of cancer in the patient.

In one or more embodiments of the invention, serum or plasma is obtained from a patient with a certain type of cancer (patient A) and administered to another patient with the same or a different type of cancer (patient B) through plasma exchange. It is to be understood that the immunoglobulins with or without their corresponding soluble receptors can alternatively be purified from serum/plasma as a means of administering the components without the additional volume inherent in total serum/plasma. By way of example, an equal amount of plasma is removed from patient B prior to, or at substantially the same time as, receiving serum or plasma from patient A. The plasma removed from patient B can be given as serum or plasma to patient A or to another patient with the same or a different type of cancer (that is, patient C). It is to be understood that this approach can be repeated and administered in tandem to other patients with the same or different types of cancer. For example, serum or plasma can be obtained from a third or fourth patient (patient D or E) and administered to the first patient (patient A) in no particular set sequence. Moreover, such administration can be administered as a single dose or as repeated doses.

As described herein, immunoglobulins (for example, IgG, IgM, IgA and IgE) specific for particular cancer antigens which are missing in one patient (for example, patient B) will be supplied by the serum or plasma of another same or different type of cancer patient (for example, patient A). A biological context for such a scenario is that when an individual is identified as having cancer, such as, for example, pancreatic cancer, it is understood that the immune system of the individual is engaged in trying to eradicate the cancer in that same individual. Unfortunately, the cancer is often not sufficiently recognized as “non-self” to be destroyed by the body's own immune system. However, the anti-cancer machinery, particularly immunoglobulins, which are raised against the cancer may be used to recognize and attack the cancer of another (second) individual because in the second individual the tumor would be seen as sufficiently foreign to invoke a proper immune response. This can be considered a type of hyperimmune anti-cancer serum/plasma which has been raised in one host of the same species with a given cancer which is sufficient to recognize subtle cancer proteins as non-self (cancer antigens) in the receiving host with having same or different type of cancer.

The serum and/or plasma of patient B that contains immunoglobulins that do not recognize cancer antigens from the same patient (patient B) can recognize other distinct antigens of the same type of cancer which are present in another patient (for example, patient A or patient C). In such a context, serum/plasma obtained from one person with a given type of cancer can contain hyperimmune anti-cancer antibodies which can recognize and destroy cancer in another patient with the same, or even a different, type of cancer. In this manner, patients with the given type of cancer can both provide and receive serum/plasma which contain hyperimmune anti-cancer antibodies can recognize distinct anti-cancer antigens/proteins in individuals with the same or different type(s) of cancer to thereby effect treatment of the cancer.

As such, one or more embodiments of the invention will facilitate and enable the reconstitution of the patient's humoral immune system to destroy cancer cells and alleviate tumor progression. As stated above, it is to be appreciated that the immunoglobulins with or without their corresponding soluble receptors can alternatively be purified from serum/plasma as a means to administer the components without the additional volume inherent in total serum/plasma.

By way of illustration only and without loss of generality, in pancreatic cancer, the roles of immunoglobulin E (IgE) and CD23 (also known as FcεRII, which is a “low affinity” receptor for IgE) in pancreatic cancer have not, thus far, been investigated as a diagnostic indicator and/or therapeutic intervention for this deadly disease. As detailed herein, IgE levels can have a protective effect in pancreatic cancer and serve as a potent anti-cancer immunoglobulin. IgE is specific to pancreatic cancer antigens and can kill cancer cells by antibody-dependent cell-mediated cytotoxicity (ADCC) as well as mediate killing cancer cells by Complement (C)-mediated cytotoxicity. One or more embodiments of the present invention illustrate that immunoglobulins such as, for example, IgG, IgM, IgA and IgE and/or their respective receptors can provide a therapeutic effect in pancreatic cancer patients.

IgE metabolism is unique in that it does not follow many of the biological pathways shared among other immunoglobulin isotypes. IgE has a molecular weight of about 180,000, which is heavier than the other isotypes such as IgG, due to the fact that IgE can be heavily glycosylated. IgE is differentially synthesized by B cells through interleukin (IL)-4 dependent (Th2 type cytokine) and independent class switching signaling mechanisms. IgE metabolism is also regulated through its low affinity receptor, FcεRII (CD23).

CD23 is found on B and T lymphocytes, NK cells, monocytes, follicular dendritic cells, eosinophils and platelets, and can be shed from the cell surface by auto-cleavage to yield IgE binding factor (IgE-BF). CD23 is upregulated on certain immune cells by IL-4 and downregulated by IFNγ. CD23 also functions as an adhesion molecule, interacts with CD21 (complement receptor II), and downregulates IgE production following its cytophilic binding to IgE Fc. Furthermore, CD23 is found in two isoforms, of which the A isoform mediates endocytosis of soluble IgE complexes, and the B isoform plays a role in antigen presentation to thymus cells (T cells).

The soluble form of CD23, sCD23, also known as IgE-BF, has multiple effects. It is a potent regulator of IgE synthesis, perpetuating its production, and is considered a differentiation factor for early thymocytes in promoting T cell growth, signals differentiation of germinal B cells and myeloid cell precursors. sCD23 can also inhibit monocyte migration.

sCD23/CD23 has been shown to be useful markers for malignancy. For example, studies in chronic lymphocytic leukemia (CLL) and AIDS-associated lymphoma have found that elevated serum levels of sCD23 correlate with disease progression. (See, e.g., Saka B, Aldan M, Sami U, Oner D, Sanem O, Dincol G., “Prognostic Importance of Soluble CD23 in B-cell Chronic Lymphocytic Leukemia,” Clin. Lab. Haematol. 2006; 28:30-5, the disclosure of which is incorporated by reference herein in its entirety for all purposes.) Also, sCD23 is elevated in hairy cell leukemia and in the seminal plasma of infertile patients with idiopathic testicular lesions.

Although IgE is well known for its role as a mediator of allergic responses, it may, in concert with immune cells, play an important role in tumor rejection through mechanisms involving nitric oxide (NO) release. Furthermore, IgE plays a beneficial role in mediating the attack against parasitic infections through mechanisms including generation of NO and ADCC. The latter occurs when IgE binds both to the antigen (through its antibody binding fragment (Fab2) component) on the “target” cell and to its receptor, CD23 (through its antibody constant fragment (Fc) component), on the “effector” cell, such as monocytes and NK cells, and subsequently signals the effector cell to release its lytic contents, which in turn destroy the target cell.

IgE is found in trace amounts in serum when compared with IgG. Consequently, although IgG has been described as being able to mediate ADCC, IgE can be much more potent than IgG in this regard, and could be engineered to mediate a robust anti-cancer response. The ratio of IgG to IgE in normal human serum is 100 million to 1. But IgE is effective in mediating host responses despite the comparatively low concentration.

By way of example only, one or more embodiments of the invention can be prepared and/or conducted in a manner as described below.

Serum obtained from confirmed pancreatic cancer (Panc Ca) patients who have previously been shown to contain elevated levels of IgE and sCD23 were cultured with human pancreatic adenocarcinoma cells (HPAC) (targets) and peripheral blood mononuclear cells (PBMC) (effectors) for a range of 4-24 hours, wherein the greatest effect was observed at approximately 20 hours, in the presence or absence of anti-IgE or anti-IgG neutralizing antibody. In an illustrative embodiment, twelve patients were evaluated for pancreatic cancer by imaging (CT, MRI, EUS) with subsequent biopsy or surgery. Serum samples (IgG, IgM, IgA, IgE) were collected prior to any intervention. Serum IgE and sCD23 levels were measured by enzyme-linked immunosorbant assay (ELISA) in a blinded manner. Here, it is to be understood that the total levels of IgE in the confirmed cancer patients would not matter with respect to treatment using the reciprocal serum/plasma exchange methodologies of the invention described herein. Rather, it would be the levels of IgE anti-cancer antibodies that would be important—but at this point cannot be effectively measured. The invention is therefore not limited to any specific total levels of IgE.

Pancreatic cancer patient demographics included an average age of 65 with a female predominance of 3:2. The cancer stage was IIa or greater (T3NxMx) with the location of the adenocarcinoma located at the head of the pancreas. The majority of co-morbid conditions included hypertension, diabetes and end-stage renal disease (ESRD).

Serum from fifteen healthy volunteers served as controls. Patients and controls did not have any history of atopic disease or parasitic infections. IgE and sCD23 levels are expressed as international units per milliliter (IU/mL) and units per milliliter (U/mL), respectively (mean±standard error (SE)), with significance between groups set at p≦0.05 (Student's t-test).

Cytotoxicity of pancreatic cancer cells was assessed by LDH (lactate dehydrogenase) release, and its percentages were calculated through absorbance values of the LDH concentrations that were collected by microplate reader at 450-490 nanometers (nm). Data are expressed as mean percent cytotoxicity (±SE) with significance between groups set at p≦0.05 (Student's t-test).

Serum immunoglobulins (IgG, IgM, IgA) levels were detected by nephelometry (MININEPH™, commercially available from The Binding Site, Birmingham, UK) according to manufacturer's instructions. Total serum IgE and sCD23 levels were detected by Enzyme Linked ImmunoSorbent Assay (ELISA) (IgE-BioQuant; sCD23-BioSource) which was performed according to standard procedure. IgG, IgM and IgA are expressed as g/L. IgE and sCD23 levels are expressed as IU/mL and U/mL, respectively (mean±SE) with significance between groups set at p<0.05 (Student's t-test).

Pancreatic cancer cell lines, PANC-1, HPAC, and MiaPaCa-2 were obtained from American Tissue and Cell Culture (ATCC) and cultured in complete media according to ATCC guidelines in T-75 cm² flasks in a 37° C. incubator with 5% CO₂. PANC-1 and HPAC cells were grown in DMEM, 15% fetal bovine serum, 5% penicillin/streptomycin, and 0.5 mM sodium pyruvate. MiaPaCa-2 cells were grown in DMEM with Ham F-12, 5% fetal bovine serum, 5% penicillin/streptomycin, 0.002 mg/mL insulin, 0.005 mg/mL transferrin, 40 ng/mL hydrocortisone, and 10 ng/mL epidermal growth factor.

Pancreatic cancer cell lines (HPAC and PANC-1) were incubated with patient or control serum for 30 minutes. Cells were centrifuged at 1800 rpm for 10 minutes and washed three times with phosphate buffered saline (PBS). Cells were then incubated with rabbit anti-human IgE or IgG antibody for 10 minutes, followed by FITC-conjugated anti-rabbit IgG antibody for 10 minutes. Cells were centrifuged and washed with PBS between each incubation. Flow cytometric analysis was performed on a Coulter Epics XL/MCL Flow Cytometer using System II software. Specific fluorescence was reported as the percentage of cells with relative fluorescence intensity scored above background.

Pancreatic cancer cell lines HPAC, PANC-1, and MiaPaCa-2, (3-4×105 cells/mL) in appropriate media were incubated in 24 well plates at 37° C. and 5% CO₂, overnight. Media was removed and cells were fixed with 500 μL of 10% buffered formalin for 15 minutes. Formalin was removed and cells were incubated with either pancreatic cancer patient serum, normal serum (1%) or no serum in 300 μL of 1% bovine serum albumin (BSA) overnight at 4° C. Cells were then washed three times with PBS (10 minute incubation per wash) and incubated with either rabbit anti-human IgE or IgG antibody (1%) in 300 μL of 1% BSA for one hour at room temperature. Cells were washed three times with PBS and incubated with FITC-conjugated anti-rabbit antibody (0.2%) for one hour at room temperature. Cells were washed three times, re-suspended in one mL of PBS and visualized with fluorescence microscopy. Cells from representative wells were scraped, transferred to micro-slides and visualized under confocal microscopy.

Pancreatic cancer cell lines HPAC, PANC-1, and MiaPaCa-2 were grown to confluence and lysed. Briefly, 1×10⁷ cells were lysed with 1.0 mL of ice-cold RIPA buffer (150 mM NaCl, 10 mM Tris-Hcl pH 7.2, 0.1% SDS, 1.0% Triton X-100, 1% deoxycholate, 5 mM EDTA, containing 1% protease inhibitor cocktail [Sigma P8340]). Cell lysates were pipetted into a 1.5-mL microcentrifuge tube on ice, agitated for 15 minutes and centrifuged for 15 minutes at 14,000×g at 4° C. The supernatants were immediately collected into 200 μL aliquots and stored at −80° C. Prior to storage, protein quantification of the cell lysates were measured by Bradford Assay.

Cell lysates were run on 15% acrylamide gels by SDS-PAGE at differing concentrations (2-25 μL) and developed with Coomassie Blue. Subsequent analysis used 10 μL of lysates. Lysates transferred onto nitrocellulose and stained with Ponceau Red stain to visualize the lanes. Each lane was cut out as strips and de-stained with PBS. Each strip containing pancreatic cancer cell lystate was incubated individually in plastic trays with gentle rotation on an orbital shaker. Strips were first incubated with Wash Buffer (5% powdered milk/1×TBS/0.1% Tween20) for one hour at room temperature. Each strip was then incubated overnight with either pancreatic cancer patient serum, normal serum (1%) or no serum in 2 mL Wash Buffer. Strips were washed three times with PBS and incubated with rabbit anti-human IgE or IgG antibody (2%) in 2 mL Wash Buffer. Strips were washed three times in PBS and incubated with horseradish peroxidase labeled-goat anti-rabbit antibody (0.1%) in 2 mL Wash Buffer for 1.5 hours. Strips were washed three times and developed with 2 mL 3,3-diaminobenzidine tetrahydrochloride (DAB) solution.

Serum obtained from patients and controls was depleted of IgE with subsequent recovery. IgE depletion and recovery was determined by ELISA and contained no detectable IgM, IgG or IgA (nephelometry). Unfractionated, IgE depleted serum and eluted (purified) IgE were stored at −70° C. until utilized. Serum obtained from patients with pancreatic cancer were incubated with increasing amounts (1-10%) of anti-human IgE or IgG (as a comparison) for 30 minutes at room temperature prior to culture in cytotoxicity assays.

With respect to antibody dependent cell-mediated cytotoxicity and effector cells, peripheral blood mononuclear cells (PBMC) served as effector cells. Ten mL of blood was collected from a healthy volunteer and diluted 1:1 with PBS. Diluted blood was layered 1:1 on Ficoll Hypaque solution and centrifuged at 1800 rpm for 30 minutes at room temperature. The buffy coat layer, including PBMC, was transferred into fresh tubes, washed twice and re-suspended in PBS. PBMCs were re-suspended in PBS. Cell viability was >99% (trypam blue exclusion dye).

HPAC (5×10³) cells were aliquoted into each well of a 96 well plate and served as the target cells. PBMCs (2.5×10⁴) were aliquoted into each well and served as the effector cells. By way of example, 5:1 effector to target (E:T) cell ratio illustrated a preferred ratio for cytotoxicity studies. E:T mixtures in phenol red dye free RPMI 1640 complete medium (1 mM L-glutamine, 100 U/ml penicillin, 100 μg/ml streptomycin, 5% heat inactivated FBS, Gibco-BRL) were incubated at 37° C., 5% CO₂ overnight with either pancreatic cancer patient serum or normal serum (100 μL final volume).

Assays were carried out using a CytoTox 96 Non-Radioactive Cytotoxicity Assay, in accordance with manufacturer's directions. This assay measures lactate dehydrogenase (LDH) release by target cells, after conversion of a tetrazolium salt into a formazan red product. Serum (1-5%) was added to 96 well tissue culture plates containing 5:1 E:T cell mixtures (100 μL final volume) and incubated at 37° C. and 5% CO₂, for 18-20 hours. In some experiments, IgE neutralized, IgE depleted serum or purified IgE was added to cytotoxicity assays. To report maximum lysis, lysing solution (9% v/v Triton-X 100) (10 μL) was added to representative culture wells. After incubation supernatant (50 μL) was transferred to a fresh 96-well plate. Chromogenic substrate solution (50 μL) was added to each well and the plates incubated, in the dark, at room temperature (RT) for 30 minutes, after which stop solution (1M NaAc) (50 μL) was added to each well to stop the reaction. The plates were read using a 96-well plate reader at 490 nanometers (nm).

When cells were cultured without serum, cell viability, as judged by trypan blue dye exclusion, and cell recovery were >99%. It was established in preliminary studies that when E:T cells were used in ratios of 10:1 or greater, or when serum concentrations were 10% or greater, LDH release could not be accurately measured because readings obtained with the spectrophotometer were above the limits of the instrument at the requisite wave length set forth by the manufacturer of the assay kit used. Further, when aliquots of LDH containing medium were diluted, linear curves were not obtained. Therefore, cells were preferably used at 5:1, and serum was preferably used at 1%.

Data described herein represent the mean of at least 3 experiments+SE, and are expressed as percent cytotoxicity. Background controls were corrected for and included target cell spontaneous (HPACs in media), target cell maximum (HPACs in media with 10 μL lysis solution), and effector cell spontaneous (PBMCs in media). The target cell maximum was corrected for by a volume control (media with 10 μL lysis solution). Each experimental was corrected for by a serum control (serum in media). These controls were incubated on the same 96-well plate as experimental assays. Percent (%) cytotoxicity was calculated according to the general equation below:

% Cytotoxicity=((Experimental−Target cell spontaneous−Effector cell spontaneous)/(Target cell maximum−Target cell spontaneous))×100.

In connection with the preparatory techniques described above, one or more illustrative embodiments of the invention are described below.

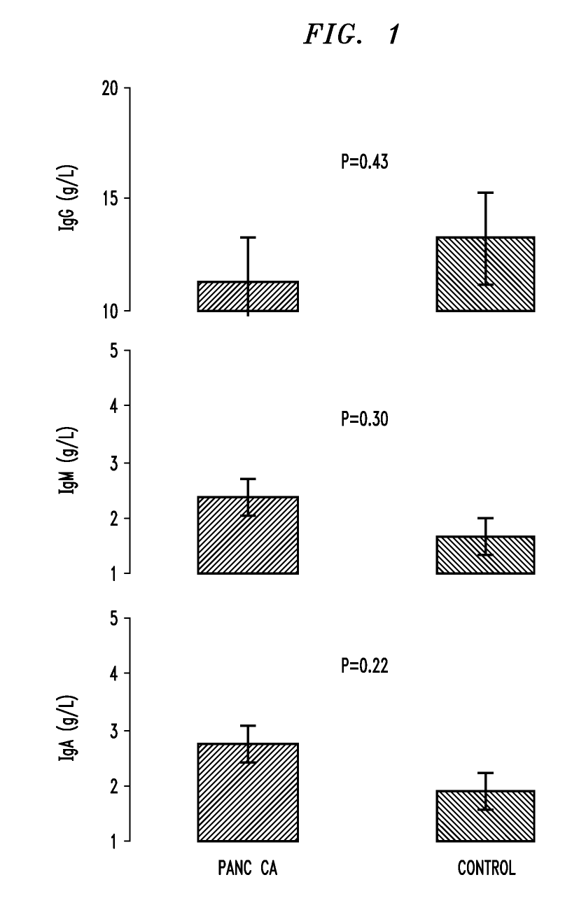

FIG. 1 is a diagram illustrating serum levels of IgG, IgM and IgA in samples with pancreatic cancer (Panc CA) versus healthy control samples without pancreatic cancer (Control), according to an embodiment of the present invention. Serum obtained from pancreatic cancer patients (n=12) and healthy controls (n=15) were assayed for IgG (top panel), IgM (middle panel), and IgA (bottom panel) by nephelometry. Data are expressed as mean g/L (±SE). Significance between groups was determined by Student's t-test (p<0.05).

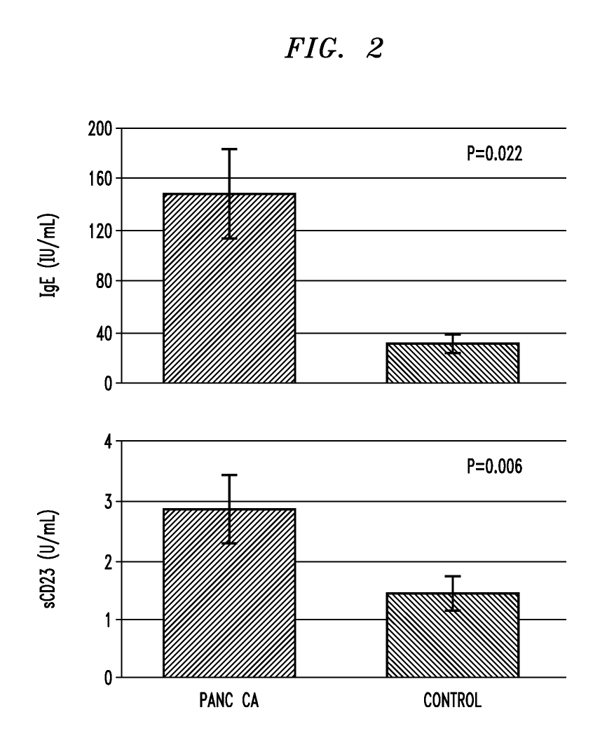

FIG. 2 is a diagram illustrating serum levels of IgE and sCD23 in samples with pancreatic cancer versus healthy control samples without pancreatic cancer, according to an embodiment of the present invention. Serum obtained from pancreatic cancer patients (n=12) and healthy controls (n=15) were assayed for IgE (top panel) and soluble CD23 (bottom panel) by ELISA. Data are expressed as mean (±SE) (IgE-IU/ml; sCD23-U/ml). Significance between groups was determined by Student's t-test (p<0.05).

As illustrated in FIG. 2, serum levels of IgE were significantly elevated in patients with pancreatic cancer (148±45 IU/ml), compared with controls (30±6.3 IU/ml) (p=0.022). In contrast, as is depicted in FIG. 1, serum levels of IgG, IgM, and IgA were similar in both pancreatic cancer and control groups (IgG: 11.2±1.2 versus 13.2±2.1, IgM: 2.39±0.8 versus 1.66±0.3, IgA: 2.72±0.7 versus 1.92+0.4; p>0.05 for all comparisons).

As also illustrated in FIG. 2, serum levels of sCD23 were also significantly elevated in patients with pancreatic cancer (2.82±0.91 U/ml) compared with controls (1.42±0.22 U/ml) (p=0.006).

The simultaneous increases of sCD23 with IgE are appropriate, in at least one aspect, because sCD23 promotes and regulates IgE production. These data illustrate a selective response for IgE regulation in pancreatic cancer. Serum levels of IgE and sCD23 were significantly increased (5-fold and 2-fold, respectively) in patients with pancreatic cancer, compared with controls.

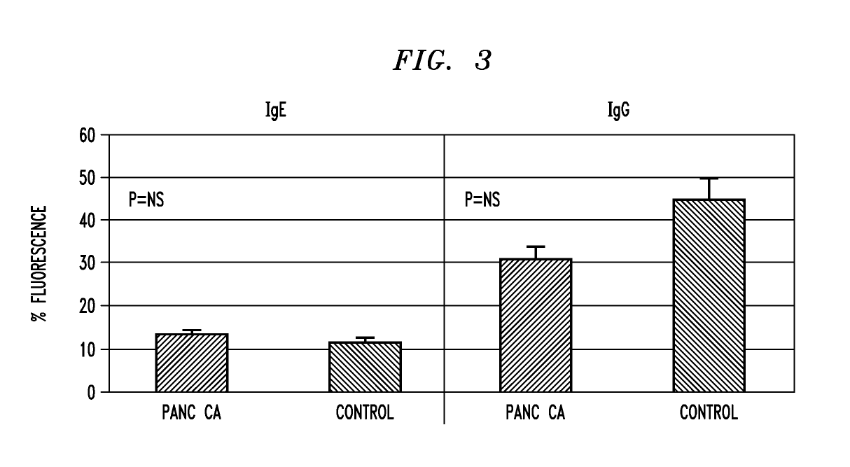

FIG. 3 is a diagram illustrating the presence of pancreatic cancer-specific immunoglobulins by immunofluorescence, according to an embodiment of the present invention. Pancreatic cancer cell lines (HPAC) were cultured in the presence of serum from pancreatic cancer patients and healthy controls. IgE and IgG in serum were detected with FITC conjugated anti-isotype immunoglobulin. Data represent mean±SD of 10 sera per group. Significance between groups was determined by Student's t-test (p=NS). Similar fluorescence patterns were detected when sera were cultured with other pancreatic cancer cell lines (MiaPaCa-2, Panc-1). Flow cytometry analysis of IgE and IgG anti-pancreatic cancer antibodies to cell surface antigens were similar between patients and controls (IgE: 12.91±0.02% vs. 11.31±0.01%, IgG: 30.98±0.15% vs. 44.81±0.28%, p>0.05) (as depicted in FIG. 3). To further establish the presence of immunoglobulin anti-cancer antibodies, pancreatic cancer cells were incubated with patient and control sera and probed for IgE and IgG. Immunofluorescence microscopy analysis also revealed the presence of anti-IgG and anti-IgE antibodies.

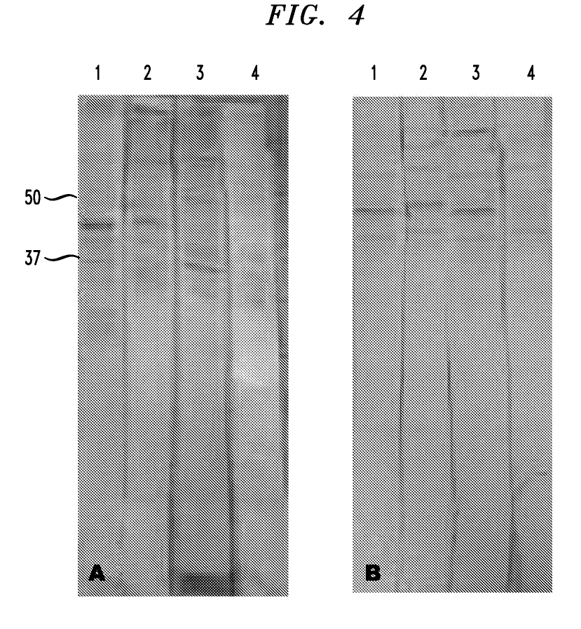

FIG. 4 is a diagram illustrating antigen-specific antibodies, according to an embodiment of the present invention. In image A (left panel), pancreatic cancer cell line (HPAC) lysates were blotted with serum obtained from pancreatic cancer patients (lanes 1 &2) or healthy controls (lanes 3&4) and probed for IgE and IgG isotypes. In image B (right panel), lysates were obtained from MiaPaCa-2 pancreatic cancer cell line and blotted with serum from pancreatic cancer patients (lanes 1-3) or healthy control (lane 4). As shown in both images, sera from patients with pancreatic cancer contained antibodies (IgG and IgE) which were specific for protein of about 50 kilodalton (kd).

In addition to cell surface recognition of IgE antibodies, the presence of specific IgE anti-pancreatic cancer antibodies against total cell components is described herein. HPAC cells were grown to confluence and lysed and the lysates were blotted and probed for the presence of antigen specific IgE. Western blot analysis revealed a 50-kd band in which was recognized by IgE obtained from pancreatic cancer patient serum which was not found in normal serum (as depicted in FIG. 4).

In order to study the function of IgE as an anti-cancer immunoglobulin, ADCC assays were performed. Serum obtained from pancreatic cancer patients mediated cytotoxicity against HPAC cell. To confirm that this effect was due to IgE, three approaches were utilized: addition of anti-IgE antibody in an effort to neutralize antibody function, depletion of IgE through immunoaffinity chromatography, and addition of purified IgE to establish that IgE alone was able to mediate cytotoxicity.

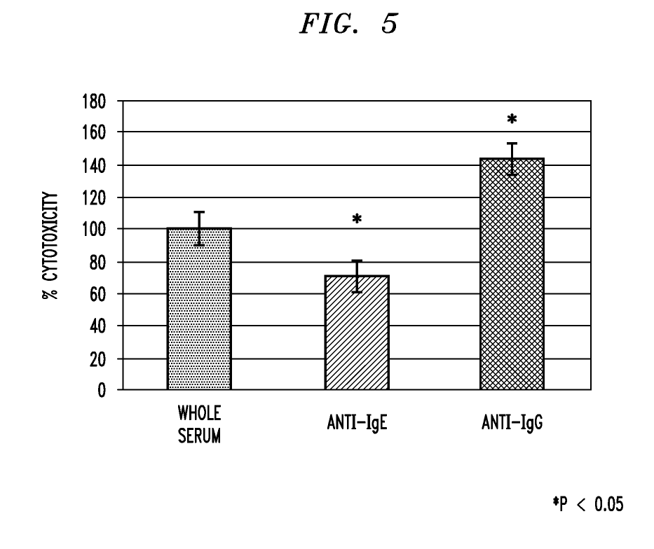

FIG. 5 is a diagram illustrating IgE mediated ADCC and effect of neutralization, according to an embodiment of the present invention. Pancreatic cancer cell lines (HPAC) were cultured in the presence of serum from pancreatic cancer patients and controls (n=4/group) and cytotoxicity was determined after 18-20 hours. In some experiments, anti-human IgE or IgG (10%) was added to serum before culture with target (HPAC) and effector (PBMC) cells. Data were performed in triplicate with an effector to target ratio of 5:1 (using 5×10³ target cells) and 1% patient serum. Data are expressed as mean percent cytotoxicity as determined by LDH release+SE of anti-IgE or IgG experiments compared with whole serum (cytotoxicity for whole serum alone averaged 17%, range 14-20%). Significance between groups was determined by Student's t-test, *p<0.05. As depicted in FIG. 5, addition of anti-IgE antibody decreased cytotoxicity by 29% when compared with whole serum. In contrast, addition of anti-IgG antibody, increased cytotoxicity by 43% (p<0.05) as shown in FIG. 5.

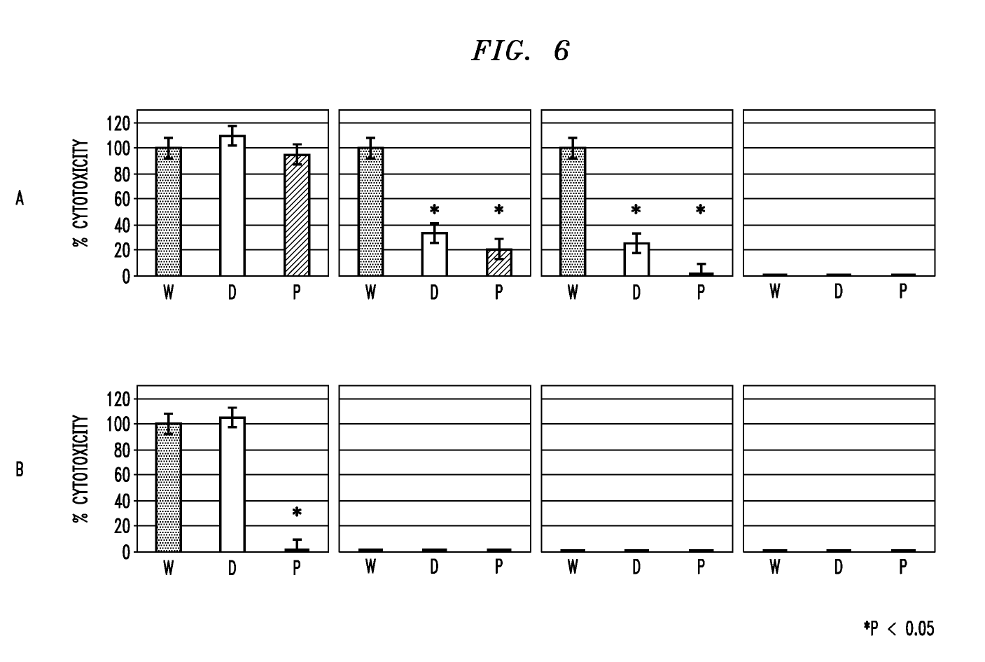

Also, each patient's serum was depleted of IgE (immunoaffinity) and depleted serum was assessed for cytotoxicity compared with unfractionated serum. FIG. 6 is a diagram illustrating IgE mediated ADCC and effect of IgE depletion and purified IgE. Pancreatic cancer cell lines (HPAC) were cultured in the presence of either whole, IgE depleted (immunoaffinity) serum or purified IgE obtained from pancreatic cancer patients in graph A and controls (n=4/group) in graph B, and cytotoxicity was determined after 18-20 hours. Experiments were performed in triplicate with an effector to target ratio of 5:1 (using 5×10³ target cells). Purified IgE contained 20-50% of original whole serum IgE content. Data are expressed as mean percent cytotoxicity as determined by LDH release+SE of anti-IgE or IgG experiments compared with whole serum (cytotoxicity for whole serum alone averaged 17%, range 14-20%). Significance between groups was determined by Student's t-test, *p<0.05. Key: W represents whole serum; D represents IgE depleted serum; and P represents purified IgE.

As shown in FIG. 6, patient serum which was depleted of IgE showed reduced cytotoxicity, whereas IgE depletion of control serum did not differ from that of unfractionated serum. Furthermore, purified IgE obtained from individual patients with pancreatic cancer was also able to mediate cytotoxicity (as depicted in FIG. 6), whereas purified IgE obtained from controls did not, demonstrating that the cytotoxicity observed with whole pancreatic cancer serum was due, in part, to IgE. In contrast, no cytotoxicity was observed when serum was cultured with target cells alone in the absence of effector cells.

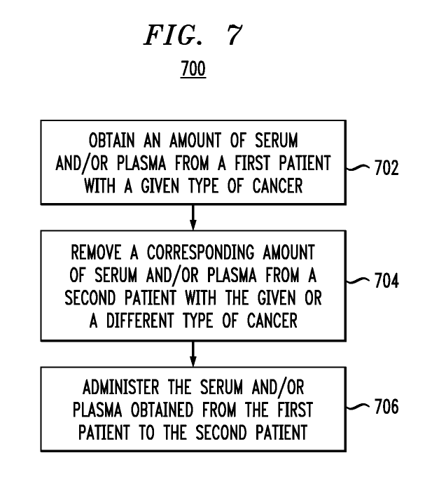

FIG. 7 is a flow diagram depicting an exemplary methodology 700 for treating cancer in a patient, according to an embodiment of the present invention. Treatment method 700 preferably begins in step 702 by obtaining an amount of serum and/or plasma from a first patient with a given type of cancer, for example pancreatic cancer. The plasma can include, for example, at least one immunoglobulin and one or more soluble receptors corresponding to each immunoglobulin. Again, it is to be understood that the immunoglobulins, with or without their corresponding soluble receptors, can alternatively be purified from serum/plasma as a means of administering the components without the additional volume inherent in total serum/plasma. The immunoglobulin may include, for example, immunoglobulin G (IgG), immunoglobulin M (IgM), immunoglobulin A (IgA) and/or immunoglobulin E (IgE). Further, the immunoglobulin can include immunoglobulin specific for one or more cancer antigens.

Optionally, method 700 may include step 704, which comprises removing a corresponding amount of serum and/or plasma from a second patient with the same type (or a different type) of cancer as the first patient. Removing the corresponding amount of serum and/or plasma from the second patient with cancer can be performed prior to or simultaneous (or nearly simultaneous) with the administration of the serum and/or plasma obtained from the first patient.

Method 700 further comprises step 706, which includes administering at least a portion of the serum and/or plasma obtained from the first patient to the second patient with the same (or different) type of cancer. Administering the serum and/or plasma obtained from the first patient to the second patient with the same (or different) type of cancer can include, for example, administering a therapeutically effective amount of an immunoglobulin and its one or more respective receptors. A therapeutically effective amount of an immunoglobulin and its one or more respective receptors may include, for example, a therapeutically effective amount of anti-cancer specific immunoglobulin.

Additionally, administering the serum and/or plasma obtained from the first patient to the second patient further can include enabling reconstitution of the second patient's humoral immune system. Enabling reconstitution of the second patient's humoral immune system can include destroying the cancer cells and/or alleviating tumor progression.

It is to be understood that, according to other embodiments of the invention, serum and/or plasma, and/or purified components obtained from such serum and/or plasma (e.g., immunoglobulins and/or their respective receptors), can be given to patients with different cancers and not just to those patients with the same cancer type. This is based, at least in part, on the understanding that many cancers arise due to an abbarency of a mechanism which is common to many types of malignancy and that these cancers develop in different types of tissues depending on cell type affected. For example, the p53 (also known as protein 53 or tumor protein 53) tumor suppressor protein is known to be responsible for many different types of cancers including, for example, liver, lung, breast and colon cancer, among other cancer types. (See, e.g., Pincus, M. R., Brandt-Rauf, P. W., Bluth M. H., Chapter 75—Oncoproteins and Early Tumor Detection, McPherson and Pincus: Henry's Clinical Diagnosis and Management by Laboratory Methods, 21st ed., Saunders Publishing, New York, August 2006, pp. 1367-1380, which is incorporated by reference herein in its entirety for all purposes).

By way of example only and without loss of generality, serum and/or plasma and/or purified components obtained from a patient with breast cancer where p53 protein abbarency is a predominant cause of the cancer, may be able to provide potent anti-p53 antibodies which will recognize and destroy cancer cells in a patient with liver cancer where p53 abbarency is also known to be a predominant cause of the cancer. It is to be understood that this methodology can be applied where the underlying cause or mechanism of the cancer is well known and even where the cause is not well known.

As detailed herein, serum IgE levels are significantly elevated in patients with cancer, such as, for example, pancreatic adenocarcinoma, and serum from these patients contain antigen specific IgE and mediate an IgE-specific cytotoxic effect against pancreatic cancer cell lines. Also, the techniques described herein include using IgE to mediate antibody dependent cell mediated cytotoxicity (ADCC). Also, as noted herein, IgE and its receptor sCD23 are elevated in serum obtained from pancreatic cancer patients, while other immunoglobulin isotypes do not differ between patient and control populations. Elevated levels of IgE and/or sCD23 may or may not necessarily be observed in patients identified as having other malignancy types. However, as previously stated, the level of IgE and/or sCD23 is not critical in the treatment methodologies according to embodiments of the invention.

Furthermore, unlike disadvantages of existing approaches, the techniques described herein demonstrate that immunoglobulins in serum and/or plasma provide anti-cancer capabilities towards the application and treatment of cancer. Optionally, serum/plasma obtained from any patient for the use of reciprocal plasma exchange methodologies according to embodiments of the invention could be irradiated prior to administration to a blood group compatible recipient. This would obviate any fear of transfusion of cancer cells from one patient to another, since irradiation would kill any passenger/bystander cells but not affect any anti-cancer immunoglobulins or respective soluble receptors.

In accordance with other embodiments of the invention, active and effective components such as anti-cancer immunoglobulins present in patients with cancer can be isolated and further amplified by synthetic and/or recombinant methods. In this manner, potent anti-cancer antibodies identified as being able to kill cancer cells obtained from an individual with cancer can be expanded to treat a plurality of individuals with the same or different cancers where the mechanisms of cancer is known (i.e, p53) or not well known. For example, an IgE anti-pancreatic cancer antibody which recognizes a particular protein epitope is identified as having anti-cancer activity (such as, for example, antibody dependent cell mediated cytotoxicity—ADCC). This particular immunoglobulin can be isolated/identified and subsequently amplified through synthetic or recombinant methods to produce substantial quantities of said agent to be administered either as a purified form or in concert with other agents or therapies to many patients with cancer to increase the effectiveness of anti-cancer treatment. Methods of agent expansion may include, but are not limited to, hybridoma-based technologies, chimeric antibody generation, in-silico modeling, and recombinant protein expression utilizing bacterial, insect or mammalian expression systems as a representation of such technologies.

Serum obtained from cancer and control patients contained IgE and IgG antibodies which recognized cell surface proteins by flow cytometry analysis and fluorescence microscopy. Also, identification of tumor-specific antigens is advantageous in formulating an effective immunotherapy in targeting tumor cells. For example, as noted herein, a 50-kd protein obtained from patients with pancreatic cancer is uniquely recognized by IgE.

One or more embodiments of the invention teach that antigen specific IgE antibodies are produced in cancer patients, such as pancreatic cancer patients, and that these antibodies can mediate anti-tumor effects through mechanisms such as, for example, ADCC, which can be an effective anti-cancer modality for immunotherapeutics. Additionally, other anti-cancer immunoglobulins (e.g., IgG, IgM, IgA), with or without their respective receptors, also possess anti-cancer activity.

As also described herein, serum from cancer patients, such as pancreatic cancer patients, were able to mediate ADCC against cancer cells. With addition of either anti-IgE neutralizing antibody, or IgE depleted serum, cytotoxic activity decreased. In contrast, when anti-IgG neutralizing antibody was added, the cytotoxic activity increased. Because IgG is found in greater quantities than IgE, neutralizing the IgG may have allowed more IgE to elicit its cytotoxic effects. The demonstration that purified IgE was able to mediate cytotoxicity alone provides further evidence for a direct IgE effect. The data indicate that the cytotoxicity observed with whole serum was mediated in part by IgE. The fact that cytotoxicity was observed in the presence of both effector and target cells and not with target cells alone supports ADCC rather than complement mediated cytotoxicity as the mechanism of action.

IgE functions by binding cytophilically to either high or low (CD23) affinity Fc receptors on various leukocytes. Furthermore, the time frame when ideal cytotoxicity was observed (e.g., 18-20 hours) suggests that PBMC effector cells or select subsets (that is, monocytes) may mediate ADCC in a manner similar to those observed in parasitic infections.

In one or more embodiments of the invention, anti-cancer effects of immunoglobulins (e.g., IgG, IgM, IgA and IgE) can be contingent on the presence and concentration of antigen-specific immunoglobulins rather than total immunoglobulin levels. The content and characteristics of antigen-specific immunoglobulins in the context of total immunoglobulin levels can vary from patient to patient. When the effect of serum mediated cytotoxicity was assessed in four individual patients and controls, 75% of patients' serum mediated cytotoxicity compared with 25% of controls. Depletion of IgE decreased the cytotoxic responses in 50% of the patients, but there were essentially no decreased cytotoxic responses in the controls. Furthermore, purified IgE mediated cytotoxicity in half of the pancreatic cancer patients but not in the controls.

In one of the patients, there were no differences in observed cytotoxicity in whole, IgE depleted serum or purified IgE. It could be that in such a patient, even though IgE was depleted >95%, sufficient levels of IgE anti-pancreatic cancer antibodies remained to mediate cytotoxicity. This would also explain why purified IgE of this patient mediated cytotoxicity comparable to whole serum. Purified IgE from such a patient contained high enough levels of IgE anti-pancreatic cancer antibodies to mediate cytotoxicity, whereas IgE purified from the serum from the other patients did not.

The observation that IgE mediated cytotoxicity was less than those reported for IgG mediated ADCC in other systems can be due to the fact that IgE is found 1000 fold less than IgG in serum concentrations. This underscores the potency of IgE as an anti-cancer agent.

Principles of the present invention illustrate that immunoglobulin anti-pancreatic cancer antibodies are present in Panc CA patients and are able to destroy pancreatic cancer cells by ADCC. The above-noted data demonstrate that IgE, sCD23, and specific anti-Panc CA antibodies provide humoral surveillance and control of malignancy, serve as useful biomarkers and are important in the immune response to the disease of pancreatic cancer. It is to be appreciated that the treatment methodologies according to embodiments of the invention can be applied to cancer in a patient in a general sense (i.e., not merely pancreatic cancer), in at least one respect, because the immune system is engaged in trying to eradicate the cancer in that same individual. However, the cancer is not sufficiently recognized as “non-self” to be adequately destroyed by the patient's own immune system. The underlying anti-cancer machinery, particularly immunoglobulins, which are raised against the cancer in one individual, however, may be beneficially used to recognize and attack cancer in another individual because, in at least one respect, in the second individual the cancer (e.g., tumor) would be seen as sufficiently foreign. This can be considered a type of hyperimmune anti-cancer serum/plasma which has been raised in one host of the same species with a given cancer which is sufficient to recognize subtle cancer proteins as non-self (cancer antigens) in the receiving host with the same, or even a different, type of cancer.

Although illustrative embodiments of the present invention have been described herein, it is to be understood that the invention is not limited to those precise embodiments, and that various other changes and modifications may be made by one skilled in the art without departing from the scope or spirit of the invention.

1. A method of treating cancer, the method comprising the steps of:
obtaining an amount of at least one of serum and plasma from at least a first patient with a given type of cancer; and
administering at least a portion of the at least one of serum and plasma obtained from the at least first patient to at least a second patient with the given type of cancer, wherein administering at least a portion of the at least one of serum and plasma obtained from the at least first patient to the at least second patient comprises: administering a therapeutically effective amount of one or more immunoglobulins and one or more soluble receptors corresponding to each immunoglobulin; and enabling reconstitution of a humoral immune system of the at least second patient to thereby treat the cancer in the at least second patient.

2. The method of claim 1, wherein the given type of cancer comprises pancreatic cancer.

3. The method of claim 1, further comprising removing a corresponding amount of at least one of serum and plasma from the second patient with the given type of cancer.

4. The method of claim 3, wherein removing the corresponding amount of at least one of serum and plasma from the second patient with the given type of cancer is performed prior to administering the at least one of serum and plasma obtained from the first patient to the second patient with the given type of cancer.

5. The method of claim 3, wherein removing the corresponding amount of at least one of serum and plasma from the second patient with the given type of cancer is performed substantially simultaneously to administering the at least one of serum and plasma obtained from the first patient to the second patient with the given type of cancer.

6. The method of claim 1, wherein the plasma and serum are interchangeable with one another with respect to treatment of the cancer in the at least second patient.

7. The method of claim 1, wherein the one or more immunoglobulins comprise at least one of immunoglobulin G (IgG), immunoglobulin M (IgM), immunoglobulin A (IgA) and immunoglobulin E (IgE).

8. The method of claim 1, wherein the one or more immunoglobulins comprise at least one immunoglobulin specific for one or more cancer antigens.

9. The method of claim 1, wherein a therapeutically effective amount of the one or more immunoglobulins and the one or more soluble receptors corresponding to each immunoglobulin comprises a therapeutically effective amount of anti-cancer specific immunoglobulin.

10. The method of claim 1, wherein enabling reconstitution of a humoral immune system of the at least a second patient comprises destroying cancer cells or alleviating tumor progression.

11. (canceled)

12. The method of claim 1, wherein administering the at least one of serum and plasma obtained from the first patient to the second patient further comprises repeatedly administering the at least one of serum and plasma obtained from the first patient to a plurality of patients with the given type of cancer.

13. The method of claim 12, wherein the at least one of serum and plasma obtained from the first patient is repeatedly administered to the plurality of patients with the given type of cancer substantially simultaneously.

14. The method of claim 1, wherein obtaining the amount of at least one of serum and plasma comprises obtaining respective amounts of at least one of serum and plasma from a plurality of patients with the given type of cancer.

15. The method of claim 14, wherein the respective amounts of at least one of serum and plasma are obtained from the plurality of patients with the given type of cancer substantially simultaneously.

16. The method of claim 1, wherein the at least one of serum and plasma obtained from the first patient is administered as at least one of a single dose and repeated doses.

17. The method of claim 1, further comprising the step of purifying the one or more immunoglobulins from the at least one of serum and plasma obtained from the at least a first patient with the given type of cancer for administering the therapeutically effective amount of the one or more immunoglobulins and the one or more soluble receptors corresponding to each immunoglobulin without additional volume associated with a total of the at least one of serum and plasma obtained from the first patient.

18. The method of claim 17, wherein the step of purifying the one or more immunoglobulins is performed prior to the step of administering the at least one of serum and plasma obtained from the first patient to the at least second patient.

19. The method of claim 1, further comprising the step of irradiating the at least one of serum and plasma obtained from the first patient.

20. The method of claim 19, wherein the step of irradiating the at least one of serum and plasma obtained from the first patient is performed prior to the step of administering the at least one of serum and plasma obtained from the first patient to the at least second patient.

21. A method of treating cancer, the method comprising the steps of:
obtaining an amount of at least one of serum and plasma from at least a first patient with a first type of cancer; and
administering at least a portion of the at least one of serum and plasma obtained from the at least first patient to at least a second patient with a second type of cancer, the second type of cancer being different than the first type of cancer, wherein administering at least a portion of the at least one of serum and plasma obtained from the at least first patient to the at least second patient comprises: administering a therapeutically effective amount of one or more immunoglobulins and one or more soluble receptors corresponding to each immunoglobulin; and enabling reconstitution of a humoral immune system of the at least second patient to thereby treat the second type of cancer.
